(19) United States
(12) Patent Application Publication
(10) Pub. No.: US 2020/0157090 A1
(43) Pub. Date: May 21, 2020

(54) NON-INVASIVE VENTILATION WITH HIGH FREQUENCY OSCILLATIONS

(71) Applicant: KONINKLIJKE PHILIPS N.V., Eindhoven (NL)

(72) Inventors: ROBERT ROMANO, PITTSBURGH, PA (US); PETER DOUGLAS HILL, MONROEVILLE, PA (US)

(21) Appl. No.: 16/749,261

(22) Filed: Jan. 22, 2020

Related U.S. Application Data
(63) Continuation of application No. 15/526,055, filed on May 11, 2017, now abandoned.
(60) Provisional application No. 62/082,302, filed on Nov. 20, 2014.

(51) Int. Cl. C07D 417/14; C07D 413/14; C07D 409/14; C07D 405/14; C07D 403/14; A01N 43/82; A01N 43/78; A01N 43/76; A01N 43/56; A01N 43/50; C07D 403/06; C07D 409/06; C07D 413/06; C07D 405/06; C07D 417/06

(52) U.S. Cl. CPC C07D 417/14; C07D 417/06; C07D 409/14; C07D 405/14; C07D 403/14; A01N 43/82; A01N 43/78; A01N 43/76; A01N 43/56; A01N 43/50; C07D 403/06; C07D 409/06; C07D 413/06; C07D 405/06; C07D 413/14

(57) ABSTRACT

The present system comprises a pressure generator, an oscillator, sensors, and computer processors. The pressure generator generates a pressurized flow of breathable gas for delivery to the airway of a subject. The oscillator causes high frequency pressure level oscillations in the pressurized flow of breathable gas. The sensors generate output signals conveying information related to one or more parameters of the gas. The computer processors receive a base expiratory pressure level and a base inspiratory pressure level and control the pressure generator and the oscillator to generate the pressurized flow of breathable gas such that during exhalation the pressure level of the pressurized flow of breathable gas oscillates from or about the base expiratory pressure level and during inhalation the pressure level of the pressurized flow of breathable gas oscillates from or about the base inspiratory pressure level.

### CROSS-REFERENCE TO RELATED APPLICATIONS

This application is a continuation of U.S. patent application Ser. No. 15/526,055, filed on May 11, 2017, which claims the priority benefit under 35 U.S.C. § 371 of International Patent Application No. PCT/IB2015/058966, filed on Nov. 19, 2015, which claims the priority benefit under 35 U.S.C. § 119(e) of U.S. Provisional Application No. 62/082,302, filed on Nov. 20, 2014, the contents of each of which are herein incorporated by reference.

### BACKGROUND OF THE INVENTION

### 1. Field of the Invention

The present disclosure pertains to a system and method for non-invasive ventilation with high frequency pressure oscillations.

### 2. Description of the Related Art

Respiratory gas exchange is impaired in many patients. Current methods of treatment include intubation of the patients, treatment with artificial lung devices, and high pressure/intensity ventilation. These measures are risky (e.g., high pressure/intensity noninvasive ventilation may damage a patient's lungs), are difficult to administer, and require extended hospitalizations with associated high costs.

### SUMMARY OF THE INVENTION

Accordingly, one or more aspects of the present disclosure relate to a system for providing respiratory therapy. The system comprises a pressure generator, an oscillator, one or more sensors, one or more physical computer processors, and/or other components. The pressure generator is configured to generate a pressurized flow of breathable gas for delivery to the airway of a subject. The oscillator is configured to cause high frequency pressure level oscillations in the pressurized flow of breathable gas. The one or more sensors are configured to generate output signals conveying information related to one or more parameters of the gas. The one or more physical computer processors are configured by computer-readable instructions to receive a base expiratory pressure level and a base inspiratory pressure level; and control the pressure generator and the oscillator to generate the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level.

Another aspect of the present disclosure relates to a method for providing respiratory therapy with a respiratory therapy system. The respiratory therapy system comprises a pressure generator, an oscillator, one or more sensors, and one or more physical computer processors. The method comprises generating a pressurized flow of breathable gas for delivery to the airway of a subject with the pressure generator; causing high frequency pressure level oscillations in the pressurized flow of breathable gas with the oscillator; generating output signals conveying information related to one or more parameters of the gas with the one or more sensors; receiving, with the one or more physical computer processors, a base expiratory pressure level and a base inspiratory pressure level; and controlling, with the one or more physical computer processors, the generation of the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level.

Still another aspect of the present disclosure relates to a system for providing respiratory therapy. The system comprises means for generating a pressurized flow of breathable gas for delivery to the airway of a subject; means for causing high frequency pressure level oscillations in the pressurized flow of breathable gas; means for generating output signals conveying information related to one or more parameters of the gas; means for receiving a base expiratory pressure level and a base inspiratory pressure level; and means for controlling the generation of the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system for non-invasive ventilation with high frequency pressure oscillations;

FIG. 2A illustrates pressure level oscillations;

FIG. 2B illustrates a non-invasive ventilation system delivering high frequency pressure oscillations;

FIG. 3 illustrates effects of oscillation during respiratory therapy; and

FIG. 4 illustrates a method for non-invasive ventilation with high frequency pressure oscillations.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

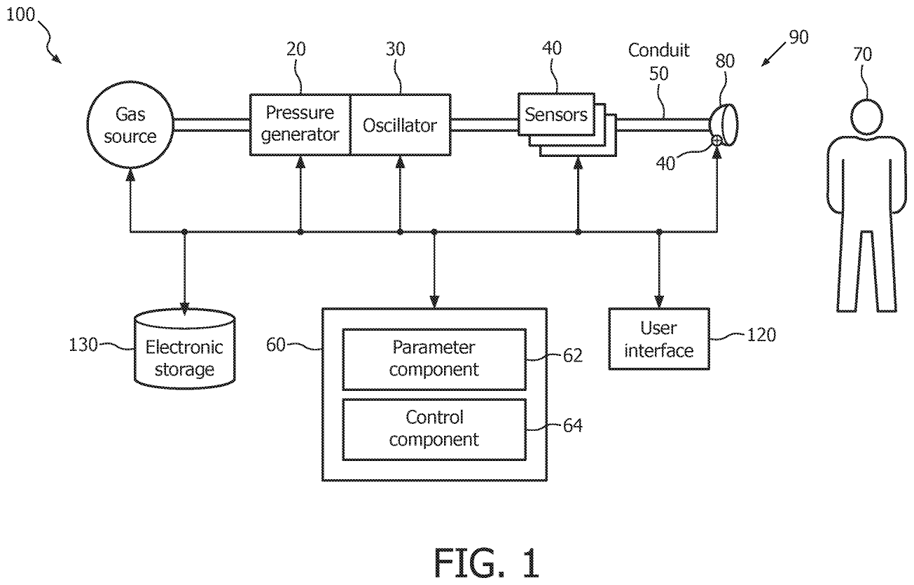

FIG. 1 is a schematic illustration of a system 100 configured to provide non-invasive ventilation with high frequency pressure oscillations to a subject 70. System 100 provides high frequency pressure oscillations simultaneously with non-invasive pressure support therapy. System 100 may be used to treat subjects 70 suffering from Chronic Obstructive Pulmonary Disease (COPD), Acute Respiratory Distress Syndrome (ARDS), Hypercapnia, Expiratory Flow Limitation (EFL), and/or other conditions. For example, patients with Hypercapnia have a high level of carbon dioxide CO₂ in their blood stream. Instead of dramatically increasing inhalation and/or exhalation therapy pressures to facilitate gas exchange, system 100 provides high frequency pressure oscillations at therapy pressure levels that are comfortable (and not dangerous) to the subject to increase diffusion of oxygen and carbon dioxide in the lungs, thus increasing CO₂ transport through the alveolar region of the lungs from the blood stream (e.g., through the tissue in the alveolar sac into the airways). System 100 may be configured to provide pressure support therapy (e.g., CPAP) with pressure levels that splint open the lower airways of subject 70 which will increase the diffusive capabilities of the high frequency oscillations into the lower alveolar regions of the lungs of subject 70. In some embodiments, system 100 comprises one or more of a pressure generator 20, an oscillator 30, one or more sensors 40, a subject interface 90, one or more physical computer processors 60, a user interface 120, electronic storage 130, and/or other components.

Pressure generator 20 is configured to generate a pressurized flow of gas for delivery to the airway of a subject 70. Pressure generator 20 may control one or more parameters of the flow of gas (e.g., flow rate, pressure, volume, temperature, gas composition, etc.) for therapeutic purposes, and/or for other purposes. By way of a non-limiting example, pressure generator 20 may be configured to control the flow rate and/or pressure of the flow of gas to provide pressure support to the airway of subject 70. Pressure generator 20 receives a flow of gas from a gas source, such as the ambient atmosphere, and elevates the pressure of that gas for delivery to the airway of subject 70. Pressure generator 20 may be any device, such as, for example, a turbine, a pump, blower, piston, or bellows, that is capable of elevating the pressure of the received gas for delivery to a patient. Pressure generator 20 may comprise one or more valves for controlling the pressure/flow of gas. In some embodiments, pressure generator 20, may generate the pressurized flow of breathable gas at pressures between about 2 cmH₂O and about 40 cmH₂O.

Oscillator 30 may be configured to cause high frequency pressure level oscillations in the pressurized flow of breathable gas. In some embodiments oscillator 30 may be and/or include one or more of a valve and/or an interrupter in the flow of the breathable gas. For example, the oscillations may be generated by oscillating the valve and/or the interrupter in the flow path of the breathable gas. In some embodiments, oscillator 30 may be included in pressure generator 20. By way of non-limiting example, pressure generator 20 may include one or more of a piston, a turbine and/or a blower that function as at least part of oscillator 30. Oscillations may be generated by oscillating one or more of the turbine, piston and/or the blower. In some embodiments, oscillator 30 may be configured to cause pressure level oscillations at a frequency of between about 2 Hz and about 20 Hz. In some embodiments, oscillator 30 may be configured to cause pressure level oscillations at a frequency of between about 3 and about 8 Hz. In some embodiments, oscillator 30 may be configured to cause pressure oscillations in the pressurized flow of breathable gas with peak to peak pressure amplitudes of between about 1 cmH₂O and about 15 cmH₂O. In some embodiments, oscillator 30 may be configured to cause pressure oscillations in the pressurized flow of breathable gas with peak to peak pressure amplitudes of between about 2 and about 8 cmH₂O. The frequencies and amplitudes described above are not intended to be limiting. The oscillations caused by oscillator 30 may have any frequency and/or any amplitude that allows system 100 to function as described herein.

Sensors 40 are configured to generate output signals conveying information related to one or more parameters of the gas within system 100. The one or more parameters of the gas within system 100 may comprise gas parameters related to the pressurized flow of breathable gas, breathing parameters related to respiration of subject 70, oscillation parameters, physiological parameters of subject 70, and/or other parameters. The one or more gas parameters of the pressurized flow of breathable gas may comprise, for example, one or more of a flow rate, a volume, a pressure, humidity, temperature, acceleration, velocity, and/or other gas parameters. Breathing parameters related to the respiration of subject 70 may comprise a tidal volume, a timing (e.g., beginning and/or end of inhalation, beginning and/or end of exhalation, etc.), a respiration rate, a duration (e.g., of inhalation, of exhalation, of a single breathing cycle, etc.), respiration frequency, and/or other breathing parameters. Oscillation parameters may comprise an oscillation frequency, an oscillation amplitude, and/or other parameters. Physiological parameters may include oximetry parameters, a pulse, a heart rate, a temperature, a blood pressure, and/or other physiological parameters.

Sensors 40 may comprise one or more sensors that measure such parameters directly (e.g., through fluid communication with the flow of gas in subject interface 90). Sensors 40 may comprise one or more sensors that generate output signals related to the one or more parameters indirectly. For example, sensors 40 may comprise one or more sensors configured to generate an output based on an operating parameter of pressure generator 20 (e.g., patient flow and/or pressure estimations from motor current, voltage, rotational velocity, and/or other operating parameters), and/or other sensors. Although sensors 40 are illustrated in FIG. 1 at a single location in system 100, this is not intended to be limiting. Sensors 40 may comprise sensors disposed in a plurality of locations, such as for example, at various locations within (or in communication with) a conduit 50, within pressure generator 20, within (or in communication with) subject interface 90, and/or other locations.

Subject interface 90 is configured to communicate the pressurized flow of breathable gas to the airway of subject 70. As such, subject interface 90 comprises conduit 50, interface appliance 80, and/or other components. In some embodiments, conduit 50 is configured to convey the pressurized flow of gas to interface appliance 80. Interface appliance 80 is configured to deliver the flow of gas to the airway of subject 70. In some embodiments, interface appliance 80 is configured to be non-invasively engaged by subject 70. Non-invasive engagement comprises removably engaging one or more external orifices of the airway of subject 70 (e.g., nostrils and/or mouth) to communicate gas between the airway of subject 70 and interface appliance 80. In some embodiments, interface appliance 80 is removably coupled to conduit 50. Interface appliance 80 may be removed for cleaning and/or for other purposes. In some embodiments, conduit 50 is configured as a mouthpiece to be engaged by the mouth of subject 70.

In some embodiments, other non-invasive interface appliances may be configured as interface appliance 80. Some examples of non-invasive interface appliance 80 may comprise, for example, a nasal cannula, a nasal mask, a nasal/oral mask, a full face mask, a total face mask, or other interface appliances that communicate a flow of gas with an airway of a subject. The present disclosure is not limited to these examples, and contemplates delivery of the flow of gas to the subject using any interface appliance. In some embodiments, system 100 may be connected to a classical respiratory circuit (e.g., a six foot hose) such that the classical respiratory circuit functions as subject interface 90.

Processor 60 is configured to provide information processing capabilities in system 100. As such, processor 60 includes one or more of a digital processor, an analog processor, a digital circuit designed to process information, an analog circuit designed to process information, a state machine, and/or other mechanisms for electronically processing information. Although processor 60 is shown in FIG. 1 as a single entity, this is for illustrative purposes only. In some implementations, processor 60 includes a plurality of processing units. These processing units may be physically located within the same device (e.g., pressure generator 20), or processor 60 may represent processing functionality of a plurality of devices operating in coordination.

As shown in FIG. 1, processor 60 is configured to execute one or more computer program components. The one or more computer program components may comprise one or more of a parameter component 62, a control component 64, and/or other components. Processor 60 may be configured to execute components 62 and 64 by software; hardware; firmware; some combination of software, hardware, and/or firmware; and/or other mechanisms for configuring processing capabilities on processor 60.

It should be appreciated that although components 62 and 64 are illustrated in FIG. 1 as being co-located within a single processing unit, in implementations in which processor 60 comprises multiple processing units, one or more of components 62 and/or 64 may be located remotely from the other components. The description of the functionality provided by the different components 62 and/or 64 described below is for illustrative purposes, and is not intended to be limiting, as any of components 62 and/or 64 may provide more or less functionality than is described. For example, one or more of components 62 and/or 64 may be eliminated, and some or all of its functionality may be provided by other components 62 and/or 64. As another example, processor 60 may be configured to execute one or more additional components that may perform some or all of the functionality attributed below to one of components 62 and/or 64.

Parameter component 62 is configured to receive, determine and/or obtain one or more parameters within system 100. The one or more parameters of the gas within system 100 may comprise gas parameters related to the pressurized flow of breathable gas, breathing parameters related to respiration of subject 70, oscillation parameters, physiological parameters of subject 70, and/or other parameters. The one or more gas parameters of the pressurized flow of breathable gas may comprise, for example, one or more of a flow rate, a volume, a pressure, humidity, temperature, acceleration, velocity, and/or other gas parameters. Breathing parameters related to the respiration of subject 70 may comprise a tidal volume, a timing (e.g., beginning and/or end of inhalation, beginning and/or end of exhalation, etc.), a respiration rate, a duration (e.g., of inhalation, of exhalation, of a single breathing cycle, etc.), respiration frequency, and/or other breathing parameters. In some embodiments, parameter component 62 is configured to receive, determine and/or obtain one or more parameters of the pressure support therapy delivered to subject 70. For example, in some embodiments, parameter component 62 is configured to receive a base expiratory pressure level and a base inspiratory pressure level for a positive pressure support therapy regime (described below). The base expiratory level and the base inspiratory level may be received by parameter component 62 via user interface 120 based on entry and/or selection of information related the positive pressure support therapy regime. Oscillation parameters may comprise an oscillation frequency, an oscillation amplitude, and/or other parameters. Physiological parameters may include oximetry parameters, a pulse, a heart rate, a temperature, a blood pressure, and/or other physiological parameters.

The determinations made by parameter component 62 may be used by control component 64 to control pressure generator 20 to control the pressurized flow of breathable gas delivered to subject 70, may be used to control oscillator 30, may be stored in electronic storage 130, and/or used for other uses.

Control component 64 is configured to control pressure generator 20 to generate the flow of gas in accordance with a positive pressure support therapy regime. Control component 64 is configured to control pressure generator 20 based on the output signals from sensors 40, information determined by parameter component 62, and/or based on other information. In some embodiments, this includes controlling pressure generator 20 to generate the pressurized flow of breathable gas at the base expiratory pressure level during exhalation and the base inspiratory pressure level (e.g., dictated by a positive pressure support therapy regime) during inhalation. In some embodiments, the base inspiratory pressure level may be up to about 40 cmH₂O, and the base expiratory pressure level may be up to about 20 cmH₂O. In positive airway pressure support therapy the pressurized flow of gas generated by pressure generator 20 is controlled to replace and/or compliment a patient's regular breathing. Positive airway pressure support therapy may be used to maintain an open airway in a patient so that oxygen and carbon dioxide may be exchanged more easily, requiring little and/or no effort from the patient. By way of non-limiting example, control component 64 may control pressure generator 20 such that the pressure support provided to the subject via the flow of gas comprises continuous positive airway pressure support (CPAP), bi-level positive airway pressure support (BPAP), proportional positive airway pressure support (PPAP), forced oscillation technique, and/or other types of pressure support therapy. CPAP supplies a fixed positive pressure to maintain a continuous level of positive airway pressure in a patient. BPAP provides a first inspiratory pressure (IPAP) and a second, typically lower, expiratory pressure (EPAP) for easier exhalation during ventilation.

In some therapy modes (e.g., PPAP), control component 64 may control pressure generator 20 to apply variable pressure support in which the amount of pressure delivered to the patient during inhalation and/or during exhalation is determined and delivered on a breath by breath basis. In some embodiments, control component 64 may be configured to control pressure generator 20 to temporarily drop the supplied pressure during exhalation (C-Flex) to reduce exhalation effort required by the patent.

In some embodiments, control component 64 is configured to control pressure generator 20 to deliver staged pressure support. In staged pressure support therapy, the pressure delivered by pressure generator 20 gradually increases over time. In some embodiments, control component 64 may control pressure generator 20 to switch therapy modes based on information related to the respiration of subject 70 and/or other information. For example, control component 64 may control pressure generator 64 to change from BPAP to CPAP after a certain number of breaths by subject 70.

In some embodiments, control component 64 is configured to control pressure generator 20 and oscillator 30 to generate the pressurized flow of breathable gas with high frequency pressure oscillations. In some embodiments, control component 64 is configured such that the high frequency pressure level oscillations are superimposed on the pressurized flow of breathable gas generated by pressure generator 20. Control component 64 is configured to control pressure generator 20 and oscillator 30 such that the pressurized flow of breathable gas generated by pressure generator 20 and oscillator 30 oscillates based on the received base expiratory pressure level (e.g., determined by parameter component 62 according to a positive pressure support therapy regime) during exhalation and based on the base inspiratory pressure level (determined by parameter component 62 according to the positive pressure support therapy regime) during inhalation.

In some embodiments, control component 64 may control pressure generator 20 and/or oscillator 30 to cause pressure level oscillations at a frequency of between about 0.5 and about 12 Hz. In some embodiments, pressure generator 20 and/or oscillator 30 may be controlled to cause pressure level oscillations at a frequency of between about 3 and about 8 Hz. In some embodiments, pressure generator 20 and/or oscillator 30 may be controlled to cause pressure level oscillations at a frequency of up to about 12 Hz. In some embodiments, pressure generator 20 and/or oscillator 30 may be controlled to cause pressure oscillations in the pressurized flow of breathable gas with peak to peak pressure amplitudes of between about 1 cmH₂O and about 15 cmH₂O. In some embodiments, pressure generator 20 and/or oscillator 30 may be controlled to cause pressure oscillations in the pressurized flow of breathable gas with peak to peak pressure amplitudes of between about 2 and about 8 cmH₂O. The frequencies and amplitudes described above are not intended to be limiting. The oscillations caused by pressure generator 20 and/or oscillator 30 may have any frequency and/or any amplitude that allows system 100 to function as described herein.

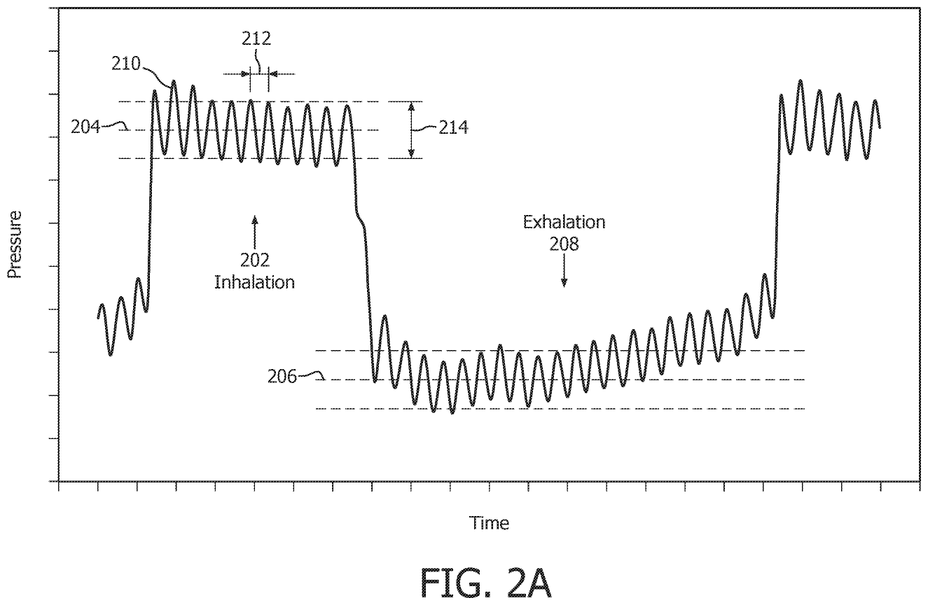

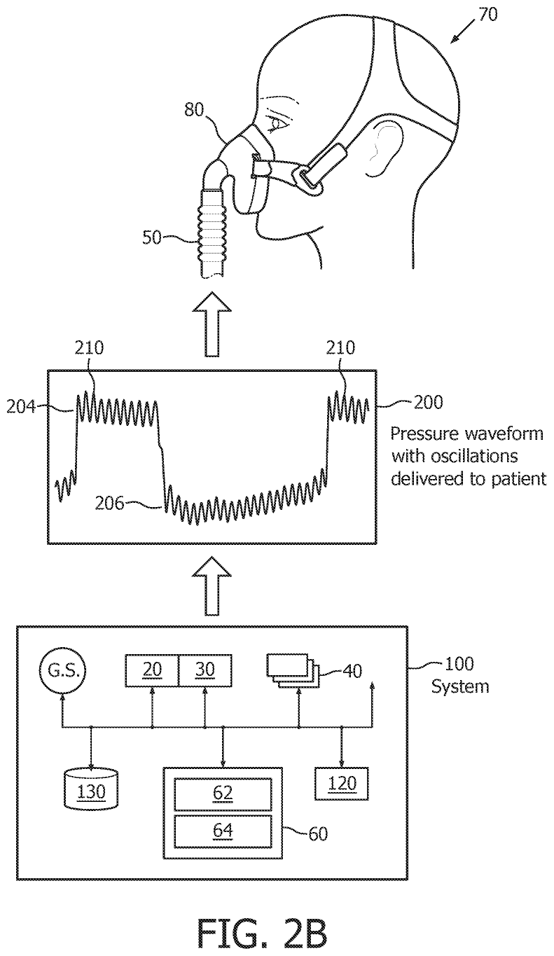

By way of non-limiting example, FIG. 2A illustrates pressure level oscillations in the pressurized flow of breathable gas. In this example, pressure level of the breathable gas oscillates from or about the base inspiratory pressure level 204 during inhalation 202, and during exhalation 208 the pressure level of the breathable gas oscillates from or about the base expiratory pressure level 206. High frequency pressure oscillations 210 are generated at a frequency 212 with a peak to peak amplitude 214. High frequency pressure oscillations oscillate based on the base inspiratory pressure level 204, and based the base expiratory pressure level 206 as shown in FIG. 2A. For example, High frequency pressure oscillation may have peak to peak pressure amplitudes of between about 1 cmH₂O and about 15 cmH₂O.

Returning to FIG. 1, in some embodiments, control component 64 is configured to detect collapse of the lower airways during exhalation. Collapse of the lower airways during exhalation may be and/or cause expiratory flow limitation (EFL). Collapse of the lower airways during exhalation may cause pulmonary gases, including CO₂, to be trapped in the alveolar region of the lungs which causes poor gas exchange and a buildup of CO₂ in the blood. Expiratory flow limitation may be detected based on the output signals, parameters determined by parameter component 62, and/or other information. For example, expiratory flow limitation (EFL) may be detected based on output signals from a pulse oximeter (e.g., included in sensors 40), an electromyogram, a pressure sensor, a flow sensor, forced oscillation technique, and/or other detection techniques. Control component 64 may be configured to automatically adjust (e.g., increase and/or decrease) the base expiratory pressure level (e.g., EPAP) responsive to detecting expiratory flow limitation. Adjusting the EPAP value may keep airway walls of subject 70 open and allow for better gas exchange such as better CO₂ removal or Oxygen transport to the circulatory system.

User interface 120 is configured to provide an interface between system 100 and subject 70 and/or other users through which subject 70 and/or other users may provide information to and receive information from system 100. Other users may comprise, for example, a caregiver, a doctor, and/or other users. This enables data, cues, results, and/or instructions and any other communicable items, collectively referred to as “information,” to be communicated between a user (e.g., subject 70) and one or more of pressure generator 20, processor 60, and/or other components of system 100. For example, a user may specify one or more therapy regimes and one or more therapy set points that are to be delivered to subject 70 using user interface 120. For example, a user may define therapy set points including a base expiratory pressure level and a base inspiratory pressure level of a positive pressure support therapy using interface 120. Control component 64 may then customize the therapy regime delivered to the subject based on the one or more inputs made by the user to the user interface. As another example, therapy pressures, the breath rate of subject 70, and/or other information may be displayed to a user (e.g., subject 70) via user interface 120. Examples of interface devices suitable for inclusion in user interface 120 comprise a keypad, buttons, switches, a keyboard, knobs, levers, a display screen, a touch screen, speakers, a microphone, an indicator light, an audible alarm, a printer, a tactile feedback device, and/or other interface devices. In one embodiment, user interface 120 comprises a plurality of separate interfaces.

It is to be understood that other communication techniques, either hard-wired or wireless, are also contemplated by the present disclosure as user interface 120. For example, the present disclosure contemplates that user interface 120 may be integrated with a removable storage interface provided by electronic storage 130. In this example, information may be loaded into system 100 from removable storage (e.g., a smart card, a flash drive, a removable disk, etc.) that enables the user(s) to customize the implementation of system 100. Other exemplary input devices and techniques adapted for use with system 100 as user interface 120 comprise, but are not limited to, an RS-232 port, RF link, an IR link, modem (telephone, cable or other). In short, any technique for communicating information with system 100 is contemplated by the present disclosure as user interface 120.

In some embodiments, electronic storage 130 comprises electronic storage media that electronically stores information. The electronic storage media of electronic storage 130 may comprise one or both of system storage that is provided integrally (i.e., substantially non-removable) with system 100 and/or removable storage that is removably connectable to system 100 via, for example, a port (e.g., a USB port, a firewire port, etc.) or a drive (e.g., a disk drive, etc.). Electronic storage 130 may comprise one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 may store software algorithms, information determined by processor 60, information received via user interface 120, and/or other information that enables system 100 to function properly. Electronic storage 130 may be (in whole or in part) a separate component within system 100, or electronic storage 130 may be provided (in whole or in part) integrally with one or more other components of system 100 (e.g., user interface 120, processor 60, etc.).

Information determined by processor 60 and/or stored by electronic storage 130 may comprise information related to respiration of subject 70, compliance, use frequency, and/or other information. The information stored by electronic storage 130 may be viewed via user interface 120, by connecting (wired and/or wireless) to a separate computer, and/or other via other methods. The information stored by electronic storage 130 may be used, for example, to adjust therapy settings, used by a doctor to make medical decisions, and/or for other uses. In some embodiments, system 100 may include a wireless transmitter (not shown) and the information determined by processor 60, the information stored by electronic storage 130, and/or other information may be communicated to a care giver, for example, over a wireless network. By way of a non-limiting example, the care giver may receive use information, patient status, and/or other information, allowing the care giver to remotely track the therapy delivered by system 100.

FIG. 2B illustrates a non-invasive ventilation system 100 delivering high frequency pressure oscillations. As shown in FIG. 2A, a pressurized flow of breathable gas 200 with oscillations 202 is being delivered to subject 70 by system 100. The breathable gas delivered to subject 70 has pressure oscillations such that during inhalation the pressure level of the breathable gas oscillates from or about the base inspiratory pressure level 204, and during exhalation the pressure level of the breathable gas oscillates from or about the base expiratory pressure level 206.

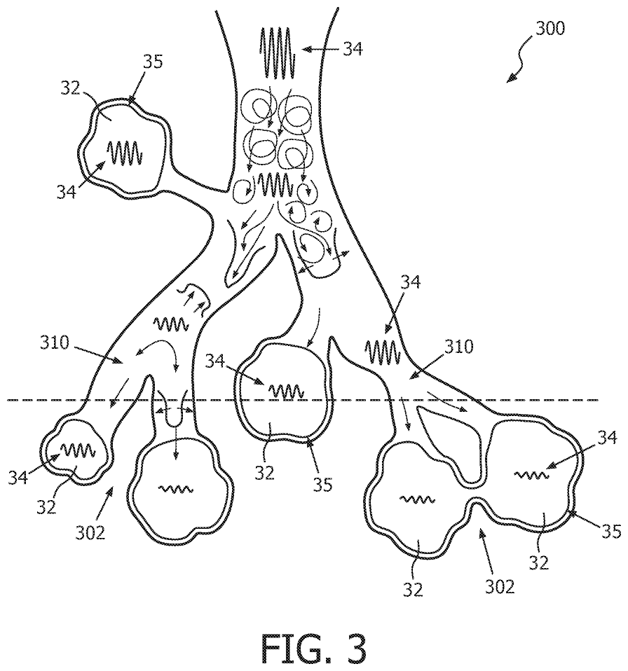

FIG. 3 illustrates effects of high frequency pressure oscillations in the lungs 300 of a subject (e.g., subject 70 shown in FIG. 1). FIG. 3 shows high frequency pressure oscillations 34 received by subject 70 that reach alveoli 32 at the far end 302 of the lungs (alveolar region) 300. High frequency pressure oscillations 34 create one or more of turbulent, penduluff, collateral, laminar airflow in lungs 300 and alveoli 32 allowing better and/or quicker O₂ and CO₂ to transport from the blood stream of the subject through the tissue in an alveolar sac 35 into airways 310. System 100 splints open the lower airways 310 and increases the diffusive capabilities of the high frequency oscillations into the lower alveolar regions at the far end 302 of lungs 300 to allow a better exchange of O₂ and CO₂.

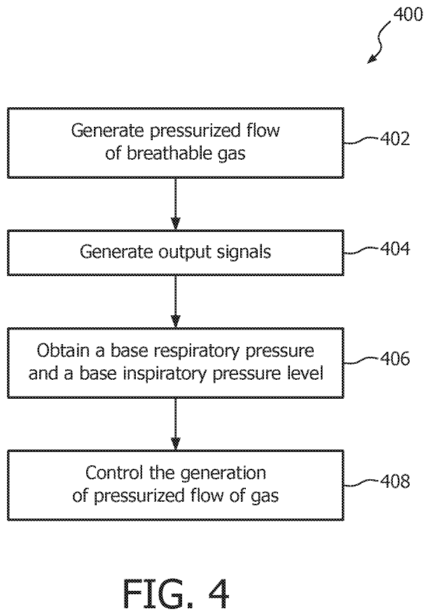

FIG. 4 illustrates a method 400 for providing respiratory therapy with a respiratory therapy system. The respiratory system comprises a pressure generator, an oscillator, one or more sensors, one or more physical computer processors, and/or other components. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

At an operation 402, a pressurized flow of breathable gas for delivery to the airway of the subject is generated. In some embodiments, the pressurized flow of breathable gas is generated by a pressure generator similar to and/or the same as pressure generator 20 (shown in FIG. 1 and described herein).

At an operation 404, output signals conveying information related to one or more parameters of the gas are generated. In some embodiments, operation 404 is performed by one or more sensors the same as or similar to sensors 40 (shown in FIG. 1 and described herein).

At an operation 406, a base expiratory pressure level and a base inspiratory pressure level are received. In some embodiments, operation 406 is performed by a physical computer processor the same as or similar to processor 60 (shown in FIG. 1 and described herein).

At an operation 408, the generation of pressurized flow of breathable gas is controlled. The control of the generation of pressurized flow of breathable gas is such that during exhalation the pressure level of the pressurized flow of breathable gas oscillates based on the received base expiratory pressure level and during inhalation the pressure level of the pressurized flow of breathable gas oscillates based on the received base inspiratory pressure level. In some embodiments, the pressure level oscillations are superimposed on the pressurized flow of breathable gas. In some embodiments the pressure level oscillations in the pressurized flow of breathable gas are caused by one or more of a valve and/or an interrupter in a flow path of the pressurized flow of breathable gas. In some embodiments operation 408 is performed by a physical computer processor the same as or similar to processor 60 (shown in FIG. 1 and described herein).

In some embodiments, method 400 includes detecting expiratory flow limitation and automatically adjusting the base expiratory pressure level responsive to detecting expiratory flow limitation. In some embodiments, detecting expiratory flow limitation is based on output signals of a pulse oximeter (e.g., included in one or more sensors 40), an electromyogram, a pressure sensor, and/or a flow sensor.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system for providing respiratory therapy, the system comprising:
a pressure generator configured to generate a pressurized flow of breathable gas for delivery to the airway of a subject;
an oscillator configured to cause high frequency pressure level oscillations in the pressurized flow of breathable gas;
one or more sensors configured to generate output signals conveying information related to one or more parameters of the gas; and
one or more physical computer processors configured by computer-readable instructions to (a) receive an input indicating a base expiratory pressure level and a base inspiratory pressure level; (b) control the pressure generator and the oscillator to generate the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level; and (c) detect expiratory flow limitation and to automatically adjust the base expiratory pressure level responsive to detecting expiratory flow limitation.

2. The system of claim 1, wherein the one or more physical computer processors are configured such that the high frequency pressure level oscillations are superimposed on the pressurized flow of breathable gas generated by the pressure generator.

3. The system of claim 1, wherein the oscillator includes one or more of a valve or an interrupter in a flow path of the pressurized flow of breathable gas.

4. The system of claim 1, wherein the one or more physical computer processors are configured such that detecting expiratory flow limitation is based on the output signals of the one or more sensors.

5. The system of claim 1, wherein the one or more physical computer processors are configured such that detecting expiratory flow limitation is based on one or more output signals of a pulse oximeter, an electromyogram, a pressure sensor, and/or a flow sensor.

6. A method of operation of a respiratory therapy system, the respiratory system comprising a pressure generator, an oscillator, one or more sensors, and one or more physical computer processors, the method comprising:
generating a pressurized flow of breathable gas with the pressure generator;
oscillating the pressurized flow of breathable gas at a high frequency;
generating output signals conveying information related to one or more parameters of the gas with the one or more sensors;
receiving an input indicating a base expiratory pressure level and a base inspiratory pressure level;
controlling the pressure generator and the oscillator to generate the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level; and
detecting expiratory flow limitation and automatically adjusting the base expiratory pressure level responsive to detecting the expiratory flow limitation.

7. The method of claim 6, wherein the high frequency oscillations are superimposed on the pressurized flow of breathable gas generated.

8. The method of claim 6, wherein the high frequency oscillations in the pressurized flow of breathable gas are caused by one or more of a valve or an interrupter in a flow path of the pressurized flow of breathable gas.

9. The method of claim 6, wherein detecting expiratory flow limitation is based on the output signals.

10. The method of claim 6, wherein detecting expiratory flow limitation is based on one or more output signals of a pulse oximeter, an electromyogram, a pressure sensor, and/or a flow sensor.

11. A system for providing respiratory therapy, the system comprising:
means for generating a pressurized flow of breathable gas for delivery to the airway of a subject;
means for causing high frequency pressure level oscillations in the pressurized flow of breathable gas;
means for generating output signals conveying information related to one or more parameters of the gas;
means for receiving an input indicating a base expiratory pressure level and a base inspiratory pressure level;
means for controlling the generation of the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level; and
means for detecting expiratory flow limitation and automatically adjusting the base expiratory pressure level responsive to detecting expiratory flow limitation.

12. The system of claim 11, wherein the high frequency pressure level oscillations are superimposed on the pressurized flow of breathable gas generated by the means for generating a pressurized flow of breathable gas.

13. The system of claim 11, wherein the means for causing high frequency pressure level oscillations include one or more of a valve or an interrupter in a flow path of the pressurized flow of breathable gas.

14. The system of claim 11, wherein the means for detecting expiratory flow limitation includes the output signals.

15. The system of claim 11, wherein the means for detecting expiratory flow limitation includes a pulse oximeter, an electromyogram, a pressure sensor, and/or a flow sensor.
